(19) United States
(12) Patent Application Publication
(10) Pub. No.: US 2014/0311929 A1
(43) Pub. Date: Oct. 23, 2014

(54) LIQUID DOSAGE FORM AND DELIVERY SYSTEM

(71) Applicant: Nupharm Laboratories Limited, Deeside (GB)

(72) Inventors: Stephen Tickle, Appleton (GB); William Michael Gould, West Kirby (GB)

(73) Assignee: Nupharm Laboratories Limited, Deeside (GB)

(21) Appl. No.: 14/255,915

(22) Filed: Apr. 17, 2014

(30) Foreign Application Priority Data
Apr. 18, 2013 (GB) 1307056.0

(51) Int. Cl. A61K 31/4439; A61J 1/00

(52) U.S. Cl. CPC A61K 31/4439; A61J 1/00
USPC 206/219; 514/338

(57) ABSTRACT

A pharmaceutical dosage form comprising solid liquid components comprising solid and liquid components; the solid component comprising omeprazole as active ingredient; one or more water soluble excipients; wherein the liquid component comprises: a surfactant; water and optional further excipients.

### CROSS-REFERENCE TO RELATED APPLICATIONS

This application claims priority under 35 U.S.C. §119 to United Kingdom Patent Application No. 1307056.0, filed 18 Apr. 2013, the disclosure of which is incorporated herein by reference in its entirety.

### DETAILED DESCRIPTION

This disclosure relates to a dosage form for a liquid pharmaceutical formulation comprising omeprazole or an enantiomer thereof including salts or other derivatives. This disclosure also relates to a delivery system for dispensing the formulation.

Omeprazole,5-methoxy-2-[[(4-methoxy-3.5-dimethyl-2-pyridinyl)methyl]sulfinyl]-1H-benzimidazole, and its alkaline salts are effective gastric acid secretion inhibitors, and are useful as antiulcer agents. The compounds being sulfoxides exist in two enantiomeric forms. The S-enantiomer is active. Omeprazole and its salts are unstable under acidic conditions, for example, below pH 7.8, and are sensitive to light, heat and moisture. Omeprazone is very hydrophobic and will not penetrate into water.

Liquid omeprazole formulations suffer from poor stability and short shelf life. The present disclosure seeks to address this problem by providing a liquid formulation which can be reconstituted immediately before administration.

According to a first aspect of the present disclosure a pharmaceutical dosage form comprises solid and liquid components;
the solid component comprising omeprazole as active ingredient; one or more water soluble excipients;
wherein the liquid component comprises: a surfactant; water and optional further excipients.

The omeprazole may comprise a racemic mixture or an enantiomer, particularly S-omeprazole. These omeprazole may be present as a salt, for example a calcium, magnesium or strontium salt or other derivative. Mixtures of omeprazole derivatives may be employed.

Typical dosages of omeprazole of 5 mg/5 cm³, 10 mg/5 cm³, 20 mg/5 cm³ or 40 mg/cm³ may be provided as an oral suspension. Alternative dosages may be provided as required.

The solid component preferably comprises a dry powder blend which is stable on storage up to 24 months.

The solid component of the disclosure preferably comprises a water soluble sweetener. Preferred excipients can be selected from the list comprising: isomalt, dextrose, lactose, maltose, maltitol, sucrose, xylitol and mixtures thereof. Preferred water soluble excipients wet easily and quickly dissolve when mixed into the liquid component. Isomalt is particularly preferred. Isomalt acts as a carrier, disintegrant sweetener and bulking agent. It has good flow properties, is stable and does not coat or stick to surfaces.

Isomalt may confer several advantages. It is non-animal in origin and has low hygroscopic properties. Isomalt exhibits excellent chemical stability, particularly having no reaction with amino groups and is resistant to degradation by enzymes and acids. It is generally regarded as a non-toxic, non-allergenic and non-irritant material, being non-cariogenic and having a low glycemic response. Isomalt is free from genetically modified organisms and has a pleasant sugar-like natural sweet taste profile.

A preferred grade of isomalt is Galen IQ720 supplied by Bene Palatinit.

The amount of isomalt may be limited by the capacity of the container but a minimum amount of 60%, preferably 70% more preferably 80% of the total weight of the solid component may be employed.

Omeprazole is bitter in taste at a low concentration of 0.2 w/v. The formulation is preferably sugar free. A combination of two or more non sugar sweeteners may be used to alleviate the bitter taste of Omeprazole. A preferred combination of sweeteners comprises a mixture of saccharine sodium, acesulfane potassium and sorbitol. Use of all three sweeteners in combination intensifies the sweetness and provides satisfactory alleviation of the bitter taste of Omeprazole. In a preferred formulation, the amounts are as follows:

| | |
|---|---|
| sodium saccharine | 0.02% to 0.5%, preferably 0.1% w/v; |
| acesulfane potassium | 0.05% to 1.0%, preferably 0.2% w/v; |
| sorbitol | 10% to 20%, preferably 14% w/v. |

The organoelectic properties may be further enhanced by addition of a flavouring, for example, 0.5% w/v strawberry flavour.

A suspending agent is preferably employed. A hydrocolloid may be used, for example a gum such as xanthan gum. An amount of about 0.01% to about 0.75% w/v may be employed. An amount of about 0.1% w/v xanthan gum has been found to be particularly effective.

The omeprazole is preferably provided in the form of granules or other solid particles which may be mixed with the water soluble excipient so that the sweetner is allowed to adhere to the excipient granules or other particles when blended. When the blend is released into the diluent the isomalt quickly absorbs water and dissolves allowing the omeprazole particles to be released and dispersed into the liquid phase. The dosage form therefore provides an oral suspension.

The liquid component may comprise water plus excipients. The excipients may be selected from the group consisting of: surfactants, antifoaming agents, buffer and biocides. Flavourings and sweeteners may be included.

The surfactant may be a non-ionic or ionic surfactant. An amount of 0.01% to 0.4% may be employed. Polysorbate 80 (polyoxyethylene (20) sorbitan monooleate) is preferred in an amount of about 0.1% to 0.5%, preferably about 0.1% to about 0.3%, more preferably about 0.2% w/v.

Percentages and other amounts used in the specification are by weight unless indicated otherwise.

A preferred biocide is domiphen bromide (N,N-dimethyl-N-(2-phenoxyethyl) dodecan-1-ammonium bromide) an amount of about 0.1% w/v is preferred. Alternative biocides may be used.

A preferred antifoaming agent is simethicone. An amount of 0.01% w/v of a 30% emulsion may be employed.

In use of the formulation the solid and liquid components are mixed with shaking as necessary to disperse the omeprazole and dissolve the soluble excipient in the liquid phase.

The buffer may be selected to provide a pH of the mixed formulation in the range 7.0 to 9.5, preferably 8.0 to 9.0. Use of an alkaline formulation is preferred in order to stabilise the omeprazole. The liquid component may have a viscosity which is sufficiently low to allow mixing by shaking but is sufficiently high to permit the omeprazole particles to be suspended prior to administration. A viscosity of 20 cP to 60 cP is preferred. A solution of 8.4% w/v sodium bicarbonate having a pH of 8.3 may be employed. The total amount of sodium bicarbonate is preferably not more than 5% w/v to avoid sedimentation after manufacture.

According to a second aspect of the present disclosure a delivery system for a dosage form in accordance with the first aspect of the present disclosure comprises:
a container having a volumetric capacity to receive the liquid component; and
a closure, adapted to releasably close the container, the closure including a receptacle for the solid component, and release means arranged when actuated to release the solid component into the container without opening the container.

Preferably the container comprises a bottle sealed with a cap, the cap including a compartment to retain the solid component and maintain it separate from the liquid component until required for use. The receptacle may include a frangible barrier or edge located between side and lower walls of the compartment which is firmly held inside and below the lip of the bottle. The patient would be required to apply reasonable force to the cap to rupture the edge of the compartment, releasing the blend into the diluent. A preferred container cap is manufactured by Rovipharm.

The container usually comprises a bottle. The bottle or other container may provide a light barrier, for example the container may be composed of amber coloured polyethylene terephthalate (PET) material. An amber or otherwise coloured glass bottle may be used. The container therefore serves to protect the reconstituted dosage forms from degradation by light.

Use of the delivery system in accordance with this disclosure confers several advantages. Individual doses of omeprazole may be provided and stored before use reducing the risk of degradation of the active ingredient. When required for use the delivery system may be activated, for example by removal of a security tab followed by release of the solid phase into the liquid phase for shaking and immediate consumption or for consumption after a period of up to 28 days. The period may be selected to allow a course of treatment to be completed using a single dosage form.

The disclosure is further described by means of example but not in any limitative sense with reference to the accompanying drawings of which:

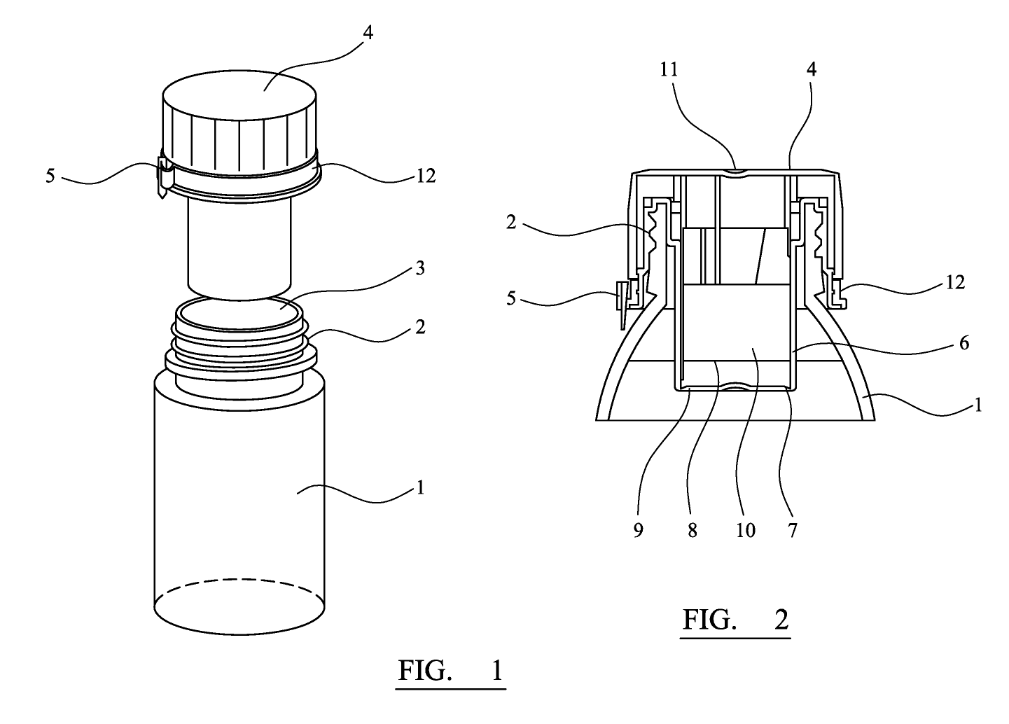

FIG. 1 illustrates the components of a delivery system before use;

FIG. 2 is a cross sectional view of an upper part of the delivery system; and

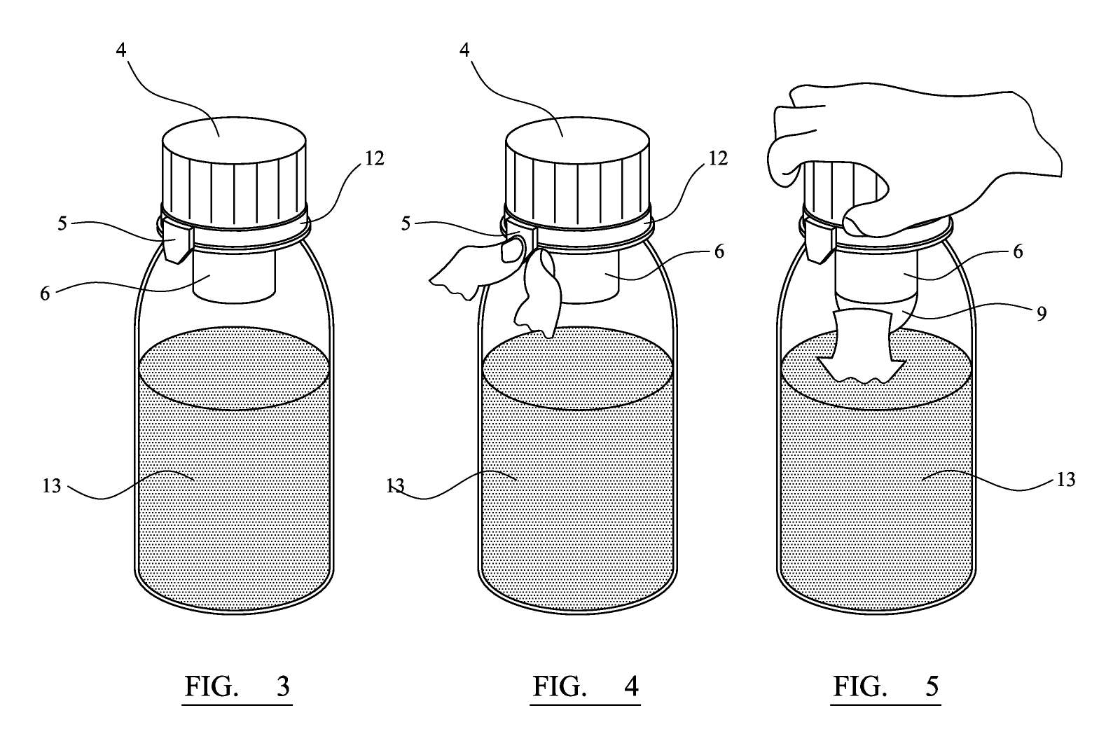

FIGS. 3 to 5 illustrate successive stages in use of the delivery system.

### EXAMPLE 1

The following ingredients were used:

### Liquid Component

| | Concentration |
|---|---|
| Raw Material | % w/v |
| Domiphen Bromide | 0.05 |
| Sodium Bicarbonate | 5.00 |
| Acesulfame K | 0.20 |
| Sodium Saccharin | 0.10 |
| Polysorbate 80 | 0.20 |
| Sorbitol 70% Solution | 20.00 |
| Simethicone 30% Emulsion | 0.02 |
| Xanthan Gum | 0.10 |
| Strawberry Flavour | 0.50 |
| Purified Water | To 100 |

Two alternative formulations were prepared

### Formulation 1—Solid Component

| | Concentration (% w/w) | | |
|---|---|---|---|
| Raw Material | 10 mg/5 ml | 5 mg/5 ml | 20 mg/5 ml |
| Omeprazole | 15.0 | 7.5 | 30 |
| Isomalt | 85.0 | 92.5 | 70 |
| (Galen IQ 720) | | | |

### Formulation 2—Solid Component

Concentration for
2 g of powder blend

| | | Mg/5 ml as a |
|---|---|---|
| Raw Materials | % w/w | suspension |
| Omeprazole | 15.0 | 10 |
| Isomalt | 85.0 | 56.7 |
| (Galen IQ 720) | | |

The following manufacturing steps were used

### Liquid Component

1. To the main vessel add 75% of the required volume of purified water.
2. Add sodium bicarbonate and stir until fully dissolved.
3. While stirring add the domiphen bromide and continue to stir until dissolved.
4. Add Simethicone 30% emulsion and Polysorbate 80 in turn and stir until fully homogeneous.
5. Add Sucralose and stir until fully dissolved.
6. Increase the stirring speed if necessary to form a vortex. Sprinkle in the xanthan gum into the top of the vortex and continue to stir at a speed high. Homogenise the batch to remove any remaining lumps of xanthan gum. Reduce the stirring speed to allow the mixture to de-aerate.
7. Add the Sorbitol 70% solution with continuous stirring until homogeneous.
8. Add the strawberry flavour and continue to stir until homogeneous.
9. Make up to volume with the remaining purified water.
10. Check the pH of the final product is between 8.0-9.0. Target pH is 8.5.
11. Fill 150 ml into 200 ml amber PET bottles.

### Solid Component

1. Weigh the ingredients into an appropriate container. Ensure that no lumps or agglomerates are present in the ingredients by sieving through a 825 micron sieve.
2. Mix the tablet core ingredients for a period of time sufficient to produce a homogeneous blend using a Turbula mixer. An amount of 2 g of the blend is used for each dosage form.

The liquid component formed a clear solution with a viscosity of 20 to 60 cP and a pH of 8.0 to 9.0. The solid component formed a white to off-white free flowing powder. The viscosity of the liquid component was selected to allow good mixing while remaining sufficiently viscous to suspend the omeprazole particles.

The absence of particles in the reconstituted suspension was tested as follows. A suspension of omeprazole 20 mg/5 ml was prepared. Using a syringe, 5 ml of the suspension was collected and attached to a 0.4 Fr Nasal Gastric (310.04) feeding tube of 40 cm. The suspension was able to travel through the tube without applying excess force to the syringe plunger.

FIGS. 1 and 2 show a delivery system for a dosage form in accordance with this disclosure.

A container (1) having screw thread (2) and cylindrical opening (3) may be engaged with a cap (4) having a tamper-proof seal (5) and a receptacle (6) extending downwardly into the container (1) through the neck (3). The receptacle (6) is generally cylindrical and has a frangible sealing disc (9) to define a container within which the solid component (10) may be stored. Upper surface (11) of the cap (4) has a downwardly extending flange (8) having a lower portion (7) extending to a point close to the frangible disc (9). When the tamper-proof ring (5) and sealing band (12) are removed the rim of the cap (4) may be depressed urging the projection (7) into contact with the frangible ring (9). This causes the ring (9) to rupture releasing the solid component (10) from the receptacle (6).

FIG. 3 shows the assembled delivery system before use wherein the liquid component (13) is disposed within the bottle (1).

In FIG. 4 the tamper proof ring (5) and sealing band (12) have been removed and the cap may then be depressed as shown in FIG. 5, releasing the solid phase (10) into the liquid component (13). The mixture of the solid component and liquid component are then shaken together before the cap is removed and the dosage form administered orally.

### EXAMPLE 2

### Nasogastric Tube Study

The use of Omeprazole 10 mg/5 ml oral suspension with nasogastric feed tubes was investigated. A sample was passed through a number 4 french nasogastric feeding tube. The number 4 french tube has the smallest internal diameter of available nasogastric tubing at approximately 1.3 mm. The sample flowed easily through the tube when a small amount of pressure was applied. The suspension does not flow through under gravity, but passed through the tubing with the aid of a syringe, with no blocking or build up of pressure observed.

The sample was tested both prior to and post passage through the nasogastric feed tube with no significant change to the concentration of omeprazole and with no observable different in the levels of related substances.

| Assay result pre Nasogastric tubing | Assay result post Nasogastric tubing |
|---|---|
| 10.01 mg/5 mL | 10.09 mg/5 mL |

### EXAMPLE 3

### Antimicrobial Activity of the Modified Formulation

Laboratory scale batches of the diluent liquid containing 80%, 90% and 100% respectively of the target preservative were prepared and analysed. The efficacy of antimicrobial preservation for each formulation at all levels satisfied the requirements of European Pharmaceopoeia

1. A pharmaceutical dosage form comprising solid liquid components comprising a solid component and a liquid component;
the solid component comprising omeprazole as active ingredient; one or more water soluble excipients;
wherein the liquid component comprises: a surfactant; water and optional further excipients.

2. The pharmaceutical dosage form as claimed in claim 1, wherein the water soluble excipient is a sweetener.

3. The pharmaceutical dosage form as claimed in claim 2, wherein the water soluble excipient is selected from the group consisting of isomalt, dextrose, lactose, maltose, maltitol, sucrose, xylitol, and mixtures thereof.

4. The pharmaceutical dosage form as claimed in claim 3 wherein the water soluble excipient is isomalt.

5. The pharmaceutical dosage form as claimed in claim 4, wherein the solid component comprises:
omeprazole 7.5%-30%; and
isomalt 70%-92.5%.

6. The pharmaceutical dosage form as claimed in claim 1, wherein the surfactant is polysorbate 80.

7. A delivery system for a dosage form comprising solid liquid components comprising a solid component and a liquid component, the delivery system comprising:
a container having a volumetric capacity to receive the liquid component; and
a closure, adapted to releasably close the container,
the closure including a receptacle for the solid component, and release means arranged when actuated to release the solid component into the container without opening the container.

8. The delivery system as claimed in claim 7 wherein the receptacle includes a frangible wall located between the capsule and the interior of the container, and means for rupturing the wall to open the receptacle and release the solid component into the container.
